(19) United States
(12) Patent Application Publication
(10) Pub. No.: US 2025/0242051 A1
(43) Pub. Date: Jul. 31, 2025

(54) Pharmaceutical Composition for Treating or Preventing Disease Caused by Reduction or Deficiency in tm5U Modification

(71) Applicant: The University of Tokyo, Tokyo (JP)

(72) Inventors: Tsutomu Suzuki, Tokyo (JP); Ena Tomoda, Tokyo (JP); Takeo Suzuki, Tokyo (JP); Asuteka Nagao, Tokyo (JP)

(21) Appl. No.: 18/575,515

(22) PCT Filed: Jul. 6, 2022
(86) PCT No.: PCT/JP2022/026887

§ 371 Date: Oct. 14, 2024

(30) Foreign Application Priority Data
Jul. 6, 2021 (JP) 2021-111955

(51) Int. Cl. A61K 48/00; A61K 31/5375; A61K 38/44; C07H 19/067; C12Q 1/6883

(52) U.S. Cl. CPC A61K 48/005; A61K 31/5375; A61K 38/443; C07H 19/067; C12Q 1/6883; C12Q 2600/156

(57) ABSTRACT

The present invention provides a pharmaceutical composition for the treatment or prevention of a disease(s) caused by reduction or deficiency in 5-taurinomethyluridine (τm⁵U) modification of mitochondrial tRNA, the composition comprising mitochondrial tRNA translation optimization 1 (MTO1) or nucleic acid encoding MTO1, wherein MTO1 is administered to a patient in an excess amount, or MTO1 is overexpressed in a patient to whom the nucleic acid has been administered.

### TECHNICAL FIELD

The present invention broadly relates, inter alia, to a pharmaceutical composition for treating or preventing a disease(s) caused by reduction or deficiency in 5-taurinomethyluridine (τm⁵U) modification of mitochondrial tRNA, and to novel applications of water-soluble carbodiimides.

### BACKGROUND ART

Mitochondrial encephalomyopathy (mitochondrial encephalomyopathy, lactic acidosis, and stroke-like episodes) (MELAS) is one of the three major types of mitochondrial diseases and characteristically presents stroke-like symptoms. Approximately 80% of MELAS patients have an A3243G mutation in the mitochondrial genome and approximately 10% of patients have a T3271C mutation. These mutations are present in the mitochondrial tRNA^{Leu(UUR)} gene.

These mutant tRNAs actually have a reduced half-life and a reduced steady-state level in the cell. In addition, it has been found that for the mutant mitochondrial tRNA^{Leu(UUR)}, the 5-taurinomethyluridine (τm⁵U) modification of the first position of the anticodon is substantially reduced, and that, of the two corresponding codons UUA and UUG, the ability to decode the UUG codon is reduced (Non-Patent Documents 1 and 2). This is a primary factor in reduction in the activity of the mitochondrial respiratory chain complex I.

It is thought, as a consequence, that mutations in the mitochondrial genome in MELAS patients impair recognition by τm⁵U-modifying enzymes (complex of MTO1 and GTPBP3).

### CITATION LIST

### Non-Patent Documents

Non-Patent Document 1: Nature Reviews Molecular Cell Biology 22 (6), 375-392 (2021)
Non-Patent Document 2: Journal of Japanese Biochemical Society 92 (3): 431-446 (2020)
Non-Patent Document 3: PNAS. 102, 7127-7132 (2005)

### SUMMARY

### Technical Problem

It has been predicted that MELAS mutant tRNA becomes structurally unstable because the mutation site is an important site for higher order structure formation in the tRNA. Due to this, it is necessary, in order to treat or prevent a disease(s) caused by reduction or deficiency in τm⁵U modification, to stabilize the structure of the mutant tRNA and restore the τm⁵U modification.

A primer extension technique has previously been used to detect τm⁵U modification. In this primer extension technique, a reverse transcription reaction is carried out using a radiolabeled DNA primer that has a sequence complementary to the RNA and the modification rate is measured utilizing the inhibition of cDNA extension due to RNA modification. It is highly sensitive and requires less than 1 μg of total RNA, making it possible to carry out an analysis with rare specimens and clinical samples.

In previous research in a laboratory with which an inventor is affiliated, τm⁵U modification was successfully detected using the primer extension technique (PE technique) and using a reverse transcriptase from Maloney murine leukemia virus (Non-Patent Document 3). However, because this technique was successful only with a specific reverse transcriptase and required that stringent reaction conditions be determined, the development of a more convenient practical technique has been desired.

### Solution to Problem

The present inventors have discovered that τm⁵U modification is recovered by an overexpression of MTO1, which constitutes a τm⁵U-modifying enzyme, in MELAS cells, and that the τm⁵U modification is conveniently detected by a reverse transcription reaction of mitochondrial tRNA^{Leu(UUR)} in the presence of a water-soluble carbodiimide.

Thus, the present invention encompasses the following inventions.
[1] A pharmaceutical composition for the treatment or prevention of a disease(s) caused by reduction or deficiency in 5-taurinomethyluridine (τm⁵U) modification of mitochondrial tRNA, the composition comprising mitochondrial RNA translation optimization 1 (MTO1) or nucleic acid encoding MTO1, wherein MTO1 is administered to a patient in an excess amount, or MTO1 is overexpressed in a patient to whom the nucleic acid has been administered.
[2] The pharmaceutical composition according to [1], wherein a τm⁵U modification rate is increased by the administration of MTO1 or the nucleic acid, in comparison to that prior to the administration.
[3] The pharmaceutical composition according to [2], wherein a τm⁵U modification rate is increased in comparison to the administration of excess GTP-binding protein 3 (GTPBP3) or in comparison to the overexpression of nucleic acid encoding GTPBP3.
[4] The pharmaceutical composition according to any of [1] to [3], wherein the nucleic acid encoding MTO1 has:
(i) nucleic acid consisting of a base sequence of SEQ ID NO: 1;
(ii) nucleic acid capable of hybridizing under stringent conditions with nucleic acid consisting of a base sequence complementary to the base sequence of SEQ ID NO: 1, and encoding protein having a τm⁵U modification activity;
(iii) nucleic acid consisting of a base sequence provided by deletion, substitution, or addition of one or a few bases in the base sequence of SEQ ID NO: 1, and encoding protein having a τm⁵U modification activity;
(iv) nucleic acid consisting of a base sequence having an at least 80% sequence identity with SEQ ID NO: 1, and encoding protein having a τm⁵U modification activity;
(v) nucleic acid consisting of a base sequence encoding protein consisting of the amino acid sequence of SEQ ID NO: 2;
(vi) nucleic acid capable of hybridizing under stringent conditions with nucleic acid consisting of a base sequence encoding protein consisting of the amino acid sequence of SEQ ID NO: 2, and encoding protein having a τm⁵U modification activity;
(vii) nucleic acid consisting of a base sequence encoding protein consisting of an amino acid sequence provided by deletion, substitution, or addition of one or a few amino acids in the amino acid sequence of SEQ ID NO: 2, with the protein having a τm⁵U modification activity; or
(viii) nucleic acid consisting of a base sequence encoding protein consisting of an amino acid sequence having a sequence identity of at least 80% with SEQ ID NO: 2, with the protein having a τm⁵U modification activity.
[5] The pharmaceutical composition according to any of [1] to [4], wherein the nucleic acid encoding MTO1 is contained in a vector.
[6] The pharmaceutical composition according to [5], wherein the vector is a viral vector or a plasmid.
[7] The pharmaceutical composition according to [6], wherein the viral vector is selected from the group consisting of retrovirus vectors, lentivirus vectors, adenovirus vectors, and adeno-associated virus vectors.
[8] The pharmaceutical composition according to any of [1] to [7], wherein the MTO1 or nucleic acid encoding MTO1 is administered systemically.
[9] The pharmaceutical composition according to any of [1] to [8], wherein the disease(s) caused by reduction or deficiency in τm⁵U modification is mitochondrial encephalomyopathy.
[10] A vector comprising nucleic acid encoding MTO1.
[12] A method of detecting τm⁵U modification of mitochondrial tRNA, the method comprising: a step of performing, in the presence of a water-soluble carbodiimide, a reverse transcription reaction on mitochondrial tRNA^{Leu(UUR)} contained in a sample derived from a subject, wherein
   the presence of τm⁵U modification is indicated when the reverse transcription reaction stops earlier than a τm⁵U at position 34 of the mitochondrial tRNA^{Leu(UUR)} or stops at a position corresponding to position 33, or
   the absence of τm⁵U modification is indicated when the reverse transcription reaction progresses to or beyond a position corresponding to position 32.
[12] The method according to [11], wherein, when τm⁵U modification is present, the reverse transcription reaction stops at a position of adenosine at position 35 or a position of uridine at position 33.
[13] The method according to or [12], wherein the reverse transcription reaction is a primer extension reaction using a reverse transcriptase.
[14] The method according to any of to [13], wherein the method further comprises a step of measuring a τm⁵U modification rate.
[15] The method according to any of to [14], wherein the mitochondrial tRNA^{Leu(UUR)} has an A3243G mutation.
[16] The method according to any of to [15], wherein the water-soluble carbodiimide is selected from the group consisting of N-cyclohexyl-N′-β-(4-methylmorpholinium) ethylcarbodiimide (CMC), 1-ethyl-3-(3-dimethylaminopropyl) carbodiimide (EDC), 4-(4,6-dimethoxy-1,3,5-triazin-2-yl)-4-methylmorpholinium chloride (DMT-MM), and salts thereof.
[17] A method of screening for drugs that treat or prevent a disease(s) caused by reduction or deficiency in τm⁵U modification, the method comprising: a step of performing, in the presence of a water-soluble carbodiimide, a reverse transcription reaction on mitochondrial tRNA^{Leu(UUR)} contained in a sample derived from a subject that has been treated with a candidate substance; and a step of measuring a τm⁵U modification rate, wherein, when the τm⁵U modification rate is increased in comparison to that for a sample where treatment with the candidate substance has not been carried out, the candidate substance is selected as a drug that treats or prevents a disease(s) caused by reduction or deficiency in τm⁵U modification.
[18] A method for in vitro detection of a disease(s) caused by reduction or deficiency in τm⁵U modification of mitochondrial tRNA, the method comprising: a step of performing, in the presence of a water-soluble carbodiimide, a reverse transcription reaction on mitochondrial tRNA^{Leu(UUR)} contained in a sample derived from a subject, wherein
   it is indicated that the subject is not suffering from a disease(s) caused by reduction or deficiency in τm⁵U modification when the reverse transcription reaction stops earlier than a τm⁵U at position 34 of the mitochondrial tRNA^{Leu(UUR)} or stops at a position corresponding to position 33, or
   it is indicated that the subject is suffering from a disease(s) caused by reduction or deficiency in τm⁵U modification when the reverse transcription reaction progresses to or beyond a position corresponding to position 32 in the mitochondrial tRNA^{Leu(UUR)}
[19] A method for determining whether or not a subject that has or is suspected of having a disease(s) caused by reduction or deficiency in τm⁵U modification is to be a target for treatment with taurine, the method comprising: a step of performing, in the presence of a water-soluble carbodiimide, a reverse transcription reaction on mitochondrial tRNA^{Leu(UUR)} contained in a sample derived from a subject, wherein
   it is indicated that the subject can be a target for a treatment using taurine, when the reverse transcription reaction stops earlier than a τm⁵U at position 34 of the mitochondrial tRNA^{Leu(UUR)} or stops at a position corresponding to position 33, or
   it is indicated that the subject can be a target for a treatment using taurine when the reverse transcription reaction progresses to or beyond a position corresponding to position 32 in the mitochondrial tRNA^{Leu(UUR)}
[20] A compound having a structure below or a salt thereof:

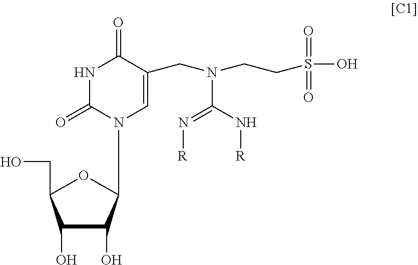

wherein R is a saturated or unsaturated hydrocarbon group, which may have a substituent, and two R's may be the same as each other.
[21] An inhibitor of a reverse transcription reaction on τm⁵U modification-comprising nucleic acid, the inhibitor comprising a water-soluble carbodiimide as an active ingredient.
[22] A kit comprising a reverse transcriptase and the reverse transcription reaction inhibitor according to [21].

### Advantageous Effects of Invention

In patients suffering from a disease(s) caused by reduction or deficiency in τm⁵U modification, the τm⁵U modification rate can be increased by the overexpression of MTO1 and mitochondrial function can then be stimulated.

In addition, τm⁵U modification can be conveniently detected by performing, in the presence of a water-soluble carbodiimide, a reverse transcription reaction on mitochondrial tRNA^{Leu(UUR)}.

### BRIEF DESCRIPTION OF DRAWINGS

FIG. 1 relates to construction of a stable MTO1-expressing strain: A) MTO1_FLAG construct; B) immunostaining of MTO1 in the stable MTO1-expressing strain.

FIG. 2 relates to RT-qPCR results for MTO1- and GTPBP3-overexpressing strains.

FIG. 3 relates to examination of taurine modification using a primer extension technique and a water-soluble carbodiimide: A) τm⁵U derivatized with CMC; B) quantitative study of τm⁵U modification rate; C) calibration curve for τm⁵U modification rate; and D, E) measurement of τm⁵U modification rate in stable overexpressing strains.

FIG. 4 relates to analysis of the steady-state level of mitochondrial tRNA^{Leu(UUR)} by Northern blotting.

FIG. 5 relates to evaluation of ability to synthesize mitochondrial proteins using a pulse-labeling technique: A) left: visualization by image analyzer of [³⁵S]-labeled mitochondrial protein, right: gel after CBB staining; B) standardization of each quantified band with wild-type strain; C) quantitation of all bands within blue frame.

FIG. 6 relates to reverse transcription reaction on mitochondrial tRNA^{Leu(UUR)}.

FIG. 7 relates to inhibition of reverse transcription reaction on mitochondrial tRNA^{Leu(UUR)} by water-soluble carbodiimide.

FIG. 8-1 shows SEQ ID NO: 1.

FIG. 8-2 is continuation of FIG. 8-1.

FIG. 9-1 shows SEQ ID NO: 2.

FIG. 9-2 is continuation of FIG. 9-1.

FIG. 9-3 is continuation of FIG. 9-2.

FIG. 9-4 is continuation of FIG. 9-3.

FIG. 9-5 is continuation of FIG. 9-4.

### DESCRIPTION OF EMBODIMENTS

Embodiments of the present invention (referred to below as the “present embodiments”) are described in the following, but the following embodiments should not be understood as limitations on the scope of the present invention.

### (The Pharmaceutical Composition)

A first embodiment provides a pharmaceutical composition for treating or preventing a disease(s) caused by reduction or deficiency in the τm⁵U modification of mitochondrial tRNA, wherein the pharmaceutical composition comprises MTO1 or nucleic acid encoding MTO1, and with which MTO1 is administered in an excess amount or MTO1 is overexpressed in a patient to whom the nucleic acid has been administered.

The τm⁵U (5-taurinomethyluridine) modification is a modification of mitochondrial tRNA and is generally seen in a wide range of animals, e.g., vertebrates (e.g., humans, mice, bovines, felines) and protochordates (ascidians) to mollusks (squid). As used herein, the “τm⁵U modification” refers to the taurine modification of mitochondrial tRNA^{Leu(UUR)}, a pos-transcriptional modification in which a taurinomethyl group is attached to the 5-position of the uracil present at position 34 (the wobble position), corresponding to the first position of anticodon of mitochondrial tRNA^{Leu(UUR)}.

A deficiency or reduction in mitochondrial tRNA modification is frequently a cause of disease. A point mutation in a tRNA gene is regarded as a cause of MELAS, which is characterized by strokes, and MERRF (myoclonic epilepsy associated with ragged-red fibers), which is characterized by epilepsy, and in such mutant mitochondrial tRNAs, taurine modification is substantially reduced and the uridine remains unmodified.

Such reduction in taurine modification can be evaluated using the proportion of τm⁵U modification at position 34 (also referred to as the τm⁵U modification rate) that is present in a specific tRNA, i.e., mitochondrial tRNA^{Leu(UUR)} As used herein, the “τm⁵U modification rate” can be given by the following formula.

$${Tm}^{5} ⋃ modification rate ( \% ) = {Tm}^{5} ⋃ / ( {Tm}^{5} ⋃ + ⋃ ) \times 100$$

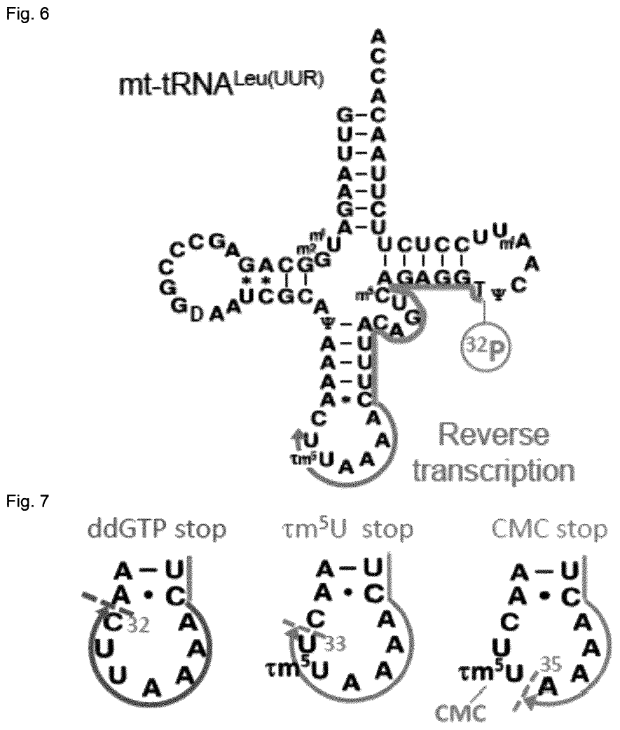

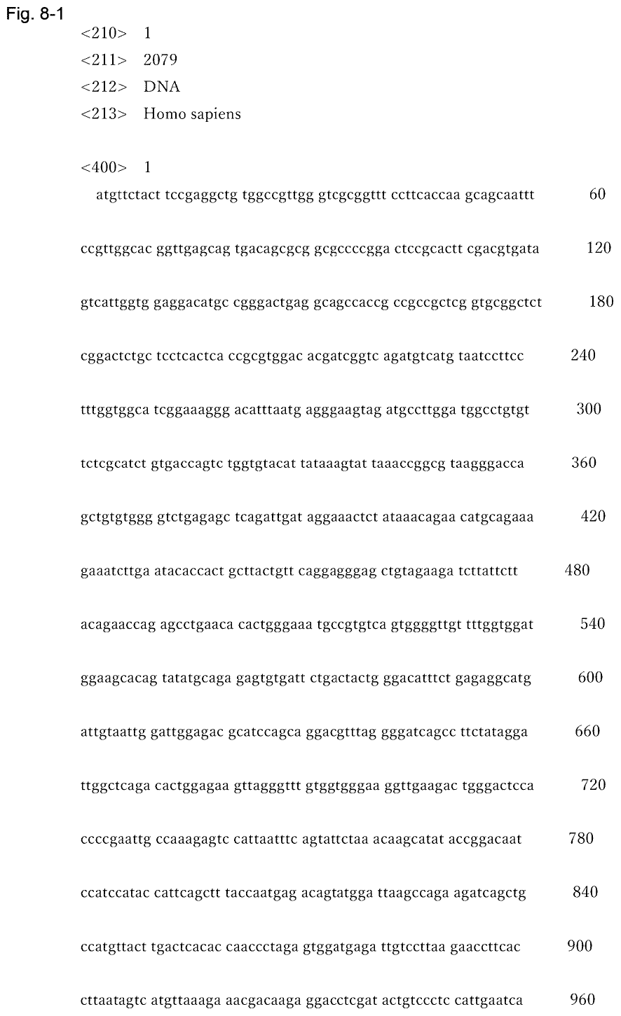

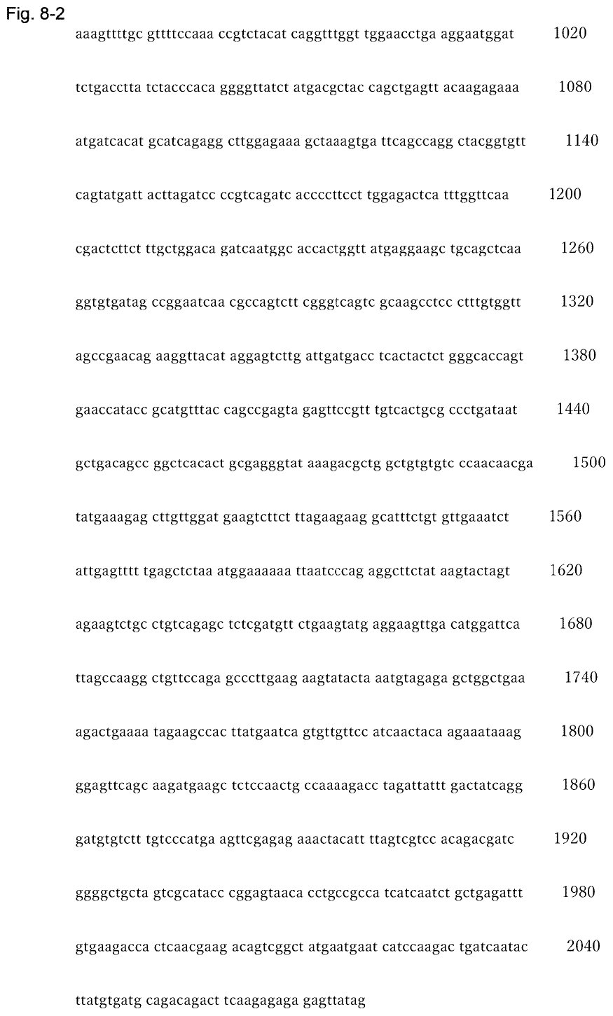

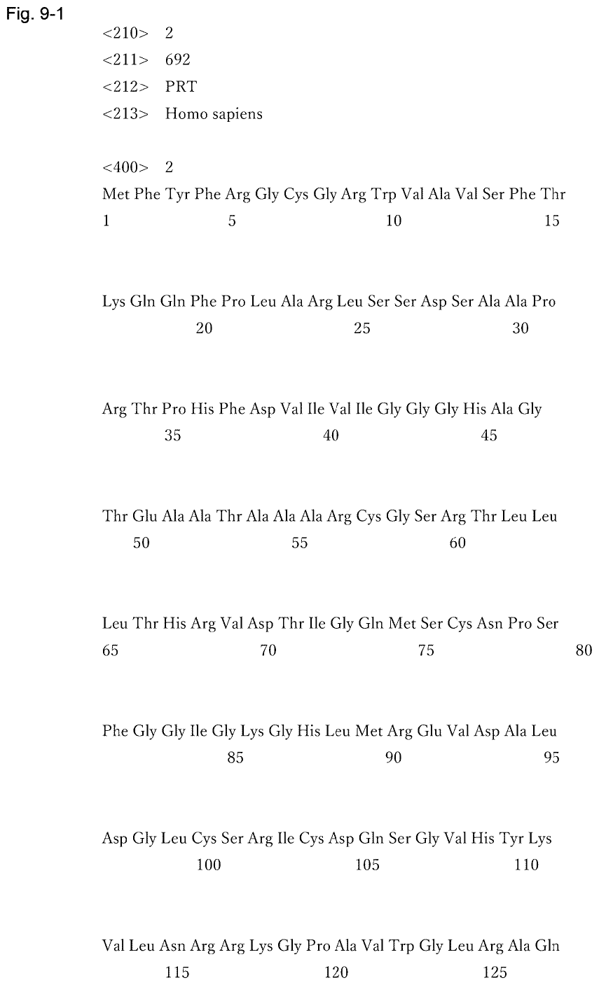

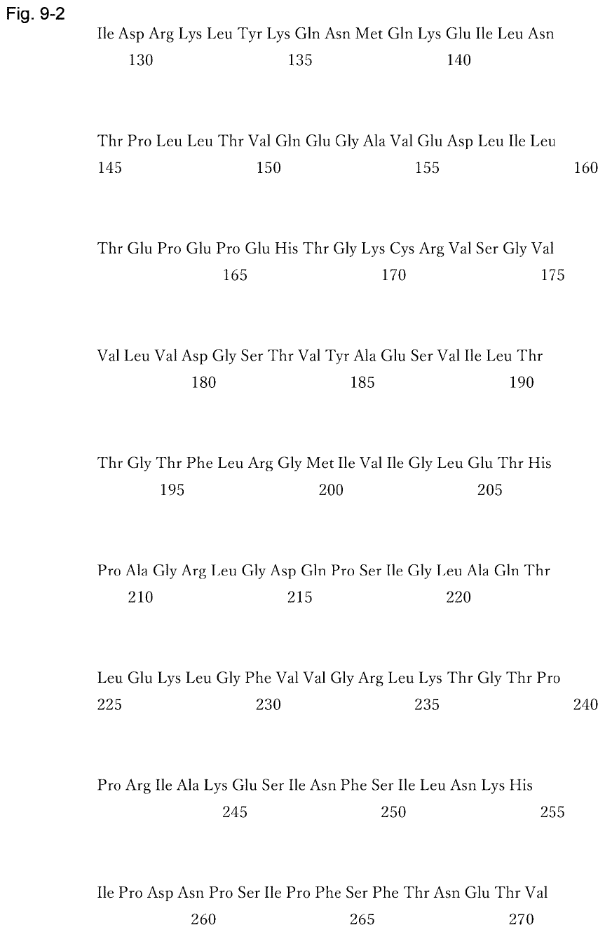

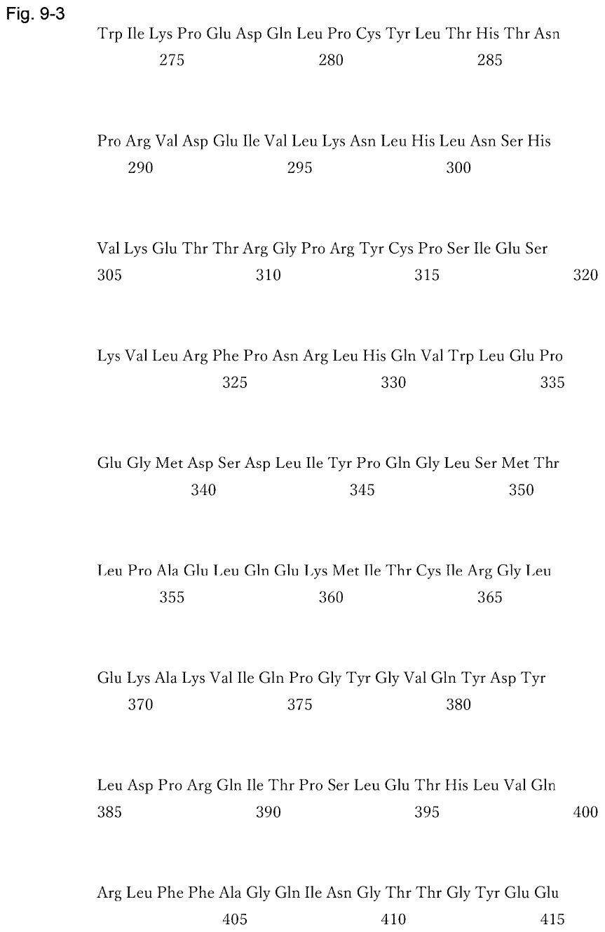

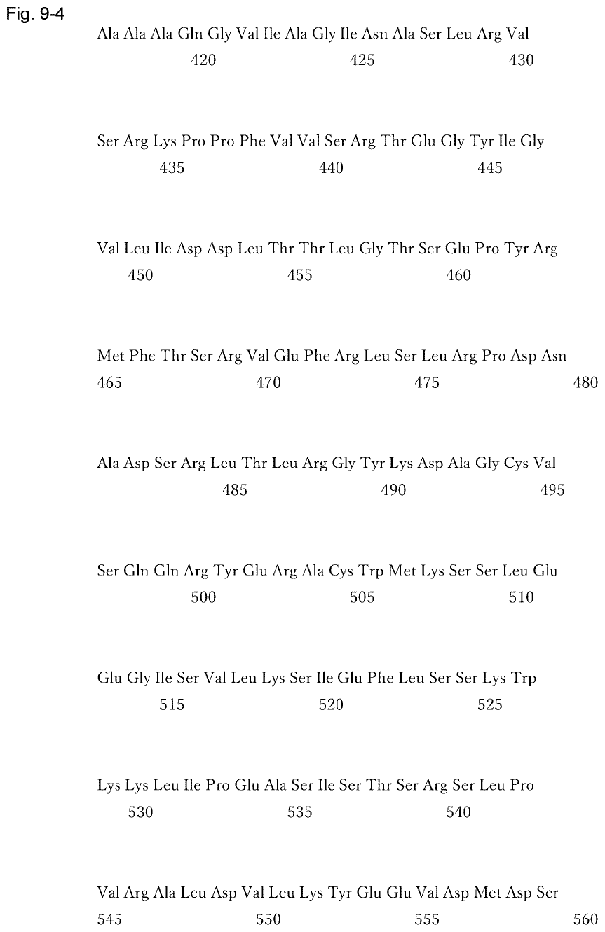

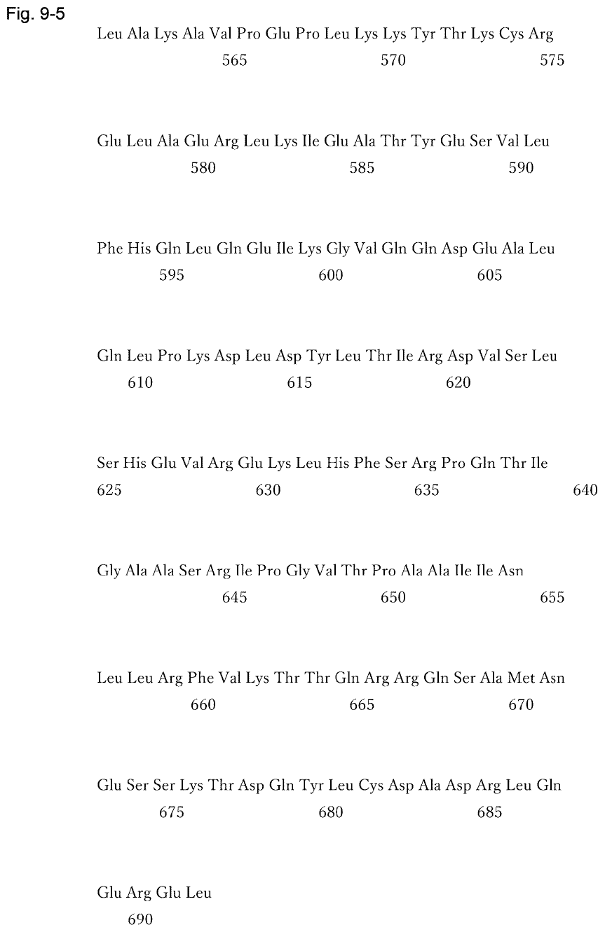

When a reverse transcription reaction is carried out on mitochondrial tRNA^{Leu(UUR)}, the reaction proceeds according to the course shown in FIG. 6, but in the case of τm⁵U modification the reverse transcription reaction stops at a position corresponding to position 33 of the mitochondria tRNA^{Leu(UUR)} (FIG. 7, center). However, when the reverse transcription reaction is carried out on a template of mitochondrial tRNA^{Leu(UUR)} in the presence of a water-soluble carbodiimide, e.g., CMC, the τm⁵U derivatized by a contact with the water-soluble carbodiimide causes the reverse transcription reaction to stop at a position corresponding to position 35 (FIG. 7, right). On the other hand, in the case of the unmodified U, the reverse transcription reaction proceeds and the extension reaction is ended by the incorporation of dideoxyguanosine triphosphate (ddGTP) at the C at position 32 (FIG. 7, left).

In the case of quantitation of the extension reaction product using, for example, denaturing polyacrylamide gel electrophoresis, the band intensities for position 32, position 33, and position 35 can be quantitated, and the band intensity ratio, as calculated using the following formula, can also be associated with the τm⁵U modification rate by constructing a calibration curve between the band intensity ratio and the τm⁵U modification rate calculated from the sample mixing ratio.

band intensity ratio=(band intensity for position 33+band intensity for position 35)/(total value of band intensities for position 32, position 33, and position 35)

As used herein, a “reduction in τm⁵U modification” means reduction in the τm⁵U modification rate, in comparison to normal tRNA^{Leu(UUR)} in which taurine modification is formed, to a degree that causes a disease such as MELAS or MERRF. For example, the τm⁵U modification rate in myoblasts from certain MELAS patients is less than 20%. However, the τm⁵U modification rate varies depending on differences in the cells, tissues, and patients. For example, it is known that the τm⁵U modification rate in Hela cells, which are commonly used cells derived from human cervical cancer, is 96.3%.

Mitochondrial encephalomyopathies, e.g., MELAS and MERRF, are examples of a disease(s) caused by reduction or deficiency in τm⁵U modification, but the present invention is not limited to these diseases.

In order to treat or prevent a disease(s) caused by reduction or deficiency in τm⁵U modification, MTO1, which is a taurine-modification enzyme, is administered in an excess amount to a patient, or MTO1 is overexpressed in a patient to whom MTO1-encoding nucleic acid has been administered.

As used herein, an “excess amount” of MTO1 and “overexpression” for MTO1-encoding nucleic acid mean a higher level in comparison to a control, for example, in comparison to the amount of MTO1 present in the body of a patient prior to the administration of the pharmaceutical composition, and means a condition that is at least approximately 1%, 5%, 25%, 50%, 75%, 100%, 125%, 150%, 175%, 200%, 250%, 300%, 350%, 400%, 450%, 500%, 600%, 700%, 800%, 900%, or at least 1000% higher than the control. 500% to at least 1000% is preferred. In a preferred embodiment, excess amount and overexpression mean a level at which, by the administration to a patient of MTO1 or MTO1-encoding nucleic acid, the τm⁵U modification rate is raised relative to prior to the administration. Thus, when MTO1 is administered directly, its excess amount is determined as appropriate in correspondence to the patient and the desired effect.

The overexpression of MTO1 can be achieved by methods well known to the person skilled in the art. For example, overexpression may be achieved by introducing an MTO1-encoding nucleic acid into the cells of a patient, for example, utilizing a lentivirus- or retrovirus-based gene transfer system, and inducing expression. When gene expression is performed using a viral gene transfer vector, the gene may be operably linked downstream from a suitable promoter, and this may be inserted into the gene transfer vector followed by transduction into cells and expression of the target gene.

As long as the desired function as MTO1 is exhibited, the MTO1 used for the administration of an excess amount of MTO1 may be a protein regarded as a fragment of MTO1 or as an MTO1 variant.

In the case of the administration of an MTO1-encoding nucleic acid to a patient for the overexpression of MTO1 in the body of the patient, the nucleic acid that is administered may be any nucleic acid capable of expressing protein having the desired function as MTO1. A vector comprising MTO1-encoding nucleic acid can be constructed, using well known methods, as a vector system that can express protein having the desired function as MTO1.

“Operably” linked indicates that, for example, the subject gene and a promoter are linked such that the desired expression, e.g., of an MTO1-encoding gene, is achieved by the promoter. A known promoter can be used. In addition, the promoter may be genetically modified to increase transcription of the MTO1-encoding gene.

When the MTO1-encoding nucleic acid is contained in a vector, the vector may be a viral vector or a plasmid. The viral vector can be selected from known vectors, for example, from the group consisting of retrovirus vectors, lentivirus vectors, adenovirus vectors, and adeno-associated virus vectors. The vector may be a self-replicating vector, for example, a plasmid, an extrachromosomal element, a minichromosome, or an artificial chromosome.

The MTO1-encoding nucleic acid may comprise the following, although this is not intended as a limitation:
(i) nucleic acid consisting of the base sequence consisting of SEQ ID NO: 1;
(ii) nucleic acid capable of hybridizing, under stringent conditions and preferably under highly stringent conditions, with nucleic acid consisting of a base sequence complementary to the base sequence of SEQ ID NO: 1, and encoding protein having a τm⁵U modification activity;
(iii) nucleic acid consisting of a base sequence provided by the deletion, substitution, or addition of one or a few bases, and preferably 1, 2, 3, 4, 5, 6, 7, 8, 9, or 10 per 100 bases, in the base sequence of SEQ ID NO: 1, and encoding protein having a τm⁵U modification activity;
(iv) nucleic acid consisting of a base sequence having a sequence identity of at least 80% and preferably at least 90%, at least 91%, at least 92%, at least 93%, at least 94%, at least 95%, at least 96%, at least 97%, at least 98%, or at least 99% with SEQ ID NO: 1, and encoding protein having a τm⁵U modification activity;
(v) nucleic acid consisting of a base sequence encoding protein consisting of the amino acid sequence of SEQ ID NO: 2;
(vi) nucleic acid encoding protein having a τm⁵U modification activity and capable of hybridizing under stringent conditions, and preferably under highly stringent conditions, with nucleic acid consisting of a base sequence encoding protein consisting of the amino acid sequence of SEQ ID NO: 2;
(vii) nucleic acid consisting of a base sequence encoding protein that comprises an amino acid sequence provided by the deletion, substitution, or addition of one or a few amino acids, and preferably 1, 2, 3, 4, 5, 6, 7, 8, 9, or 10 amino acids per 100 amino acids, in the amino acid sequence of SEQ ID NO: 2, wherein the protein has a τm⁵U modification activity; or
(viii) nucleic acid consisting of a base sequence encoding protein that comprises an amino acid sequence having a sequence identity of at least 80% and preferably at least 90%, at least 91%, at least 92%, at least 93%, at least 94%, at least 95%, at least 96%, at least 97%, at least 98%, or at least 99% with SEQ ID NO: 2, wherein the protein has a τm⁵U modification activity.

As used herein, “nucleic acid” means not only DNA, but also RNA, e.g., mRNA, or their salts.

Herein, stringent conditions refer to conditions under which a specific hybridazation is formed and nonspecific hybridazation are not formed. Stringent conditions can be readily determined by the person skilled in the art, and are generally empirical experimental conditions that depend on the base length of the nucleic acid, the washing temperature, and the salt concentration of the buffer. In general, the temperature for proper annealing is higher at a longer base length and is lower at a shorter base length. Hybridazation generally depends on the re-annealing capability in an environment in which the complementary strands are somewhat below their melting temperature.

As used herein, “highly stringent conditions” refer to conditions designed to enable the hybridization of DNA strands having a high degree of complementarity in their nucleic acid sequence and to exclude the hybridization of DNA having a significantly large number of mismatches. Stringency can vary, for example, with the concentration of the sodium chloride/sodium citrate (SSC). For example, the manual for the ECL Direct Nucleic Acid Labeling and Detection System (Amersham Pharmacia Biotech) states that a highly stringent condition can be achieved by replacing the 0.1×SSC constituting the primary wash buffer with 0.50×SSC.

The τm⁵U modification rate is substantially increased when MTO1 is administered in an excess amount to a patient or when MTO1 is overexpressed in a patient to whom MTO1-encoding nucleic acid has been administered. It is thought that this increase in the τm⁵U modification rate exceeds the increase in the τm⁵U modification rate by the administration of GTPBP3, which constitutes a τm⁵U-modifying enzyme together with MTO1, in excess or by the overexpression of a GTPBP3-encoding nucleic acid.

The dosage form, method of use, and dose of the MTO1 or MTO1-encoding nucleic acid can be determined based on general knowledge with regard to polymer drugs or nucleic acid drugs, respectively. For example, the dosage form may take the form of a parenteral formulation such as an injectable or an infusion. Carriers or vehicles that can be used in such parenteral formulations includes aqueous vehicles such as physiological saline and isotonic solutions.

The pharmaceutical composition may comprise ingredients such as pharmaceutically acceptable buffers, stabilizers, preservatives, and other additives. Pharmaceutically acceptable ingredients are well known to the person skilled in the art, and the person skilled in the art can, operating within the range of ordinary skill, select and use same according to the form of the formulation from ingredients listed in standards or norms, e.g., the Japanese Pharmacopoeia, 17th Edition.

The pharmaceutical composition can be administered orally or parenterally (locally, rectally, intravenous administration, intraarterial administration, intramuscularly, subcutaneously, and so forth). The method of administration is not particularly limited, but systemic administration by injection or infusion is preferred, for example, intravenous administration, intraarterial administration, and so forth. In another embodiment, administration to the periphery associated with the central nervous system (CNS) is also preferred. From the viewpoint of suppressing strokes in MELAS, the pharmaceutical composition is preferably introduced to the vascular endothelial cells of the brain. More preferably, it is administered to skeletal muscles systemically.

In another embodiment, there is provided a method for treating or preventing a disease(s) caused by reduction or deficiency in τm⁵U modification of mitochondrial tRNA, wherein the method comprises a step of administering MTO1 or MTO1-encoding nucleic acid to a subject wherein the MTO1 is administered in an excess amount or MTO1 is overexpressed in a patient to whom the nucleic acid has been administered.

Such a method may be carried out in combination with another therapeutic method used for the treatment or prevention of a disease(s) caused by reduction or deficiency in τm⁵U modification. For example, combination with the use of a pharmaceutical composition of, e.g., taurine, taurine chloramine, taurine precursor, tauroursodeoxycholic acid (TUDCA), and so forth, which are known as therapeutic agents for MELAS, may be considered. Among these, high-dose taurine supplementation therapy is preferred because its effectiveness in suppressing the recurrence of stroke-like attacks in MELAS has been confirmed. The MTO1 or MTO1-encoding nucleic acid may be administered simultaneously with another active ingredient, such as taurine, or may be administered at a different time. When administered simultaneously, the active ingredients may be included in the same pharmaceutical composition.

The subjects for the administration of MTO1 or MTO1-encoding nucleic acid are not limited to humans, but non-human vertebrates (e.g., monkeys, mice, rats, hamsters, guinea pigs, rabbits, cats, dogs, pigs, bovines, horses, sheep, birds, reptiles, amphibians, fish), protochordates, mollusks, and so forth that can suffer from a disease(s) caused by reduction or deficiency in τm⁵U modification are also included in this scope.

Instead of administering MTO1 or MTO1-encoding nucleic acid, or in parallel with the administration of MTO1 or MTO1-encoding nucleic acid, a treatment that enhances the expression of MTO1 in the patient can also be carried out. For example, the enhanced expression of MTO1 can be induced by enhancing the subject's own endogenous MTO1 expression activity.

### (The Vector)

A vector comprising MTO1-encoding nucleic acid is provided in a second embodiment.

The MTO1-encoding nucleic acid may be as follows:
(i) nucleic acid consisting of the base sequence consisting of SEQ ID NO: 1;
(ii) nucleic acid capable of hybridizing, under stringent conditions and preferably under highly stringent conditions, with nucleic acid consisting of a base sequence complementary to the base sequence of SEQ ID NO: 1, and encoding protein having a τm⁵U modification activity;
(iii) nucleic acid consisting of a base sequence provided by the deletion, substitution, or addition of one or a few bases, and preferably 1, 2, 3, 4, 5, 6, 7, 8, 9, or 10 per 100 bases, in the base sequence of SEQ ID NO: 1, and encoding protein having a τm⁵U modification activity;
(iv) nucleic acid consisting of a base sequence having a sequence identity of at least 80% and preferably at least 90%, at least 91%, at least 92%, at least 93%, at least 94%, at least 95%, at least 96%, at least 97%, at least 98%, or at least 99% with SEQ ID NO: 1, and encoding protein having a τm⁵U modification activity;
(v) nucleic acid consisting of a base sequence encoding protein consisting of the amino acid sequence of SEQ ID NO: 2;
(vi) nucleic acid encoding protein having a τm⁵U modification activity and capable of hybridizing under stringent conditions, and preferably under highly stringent conditions, with nucleic acid consisting of a base sequence encoding protein consisting of the amino acid sequence of SEQ ID NO: 2;
(vii) nucleic acid consisting of a base sequence encoding protein that comprises an amino acid sequence provided by the deletion, substitution, or addition of one or a few amino acids, and preferably 1, 2, 3, 4, 5, 6, 7, 8, 9, or 10 amino acids per 100 amino acids, in the amino acid sequence of SEQ ID NO: 2, wherein the protein has a τm⁵U modification activity; or
(viii) nucleic acid consisting of a base sequence encoding protein that comprises an amino acid sequence having a sequence identity of at least 80% and preferably at least 90%, at least 91%, at least 92%, at least 93%, at least 94%, at least 95%, at least 96%, at least 97%, at least 98%, or at least 99% with SEQ ID NO: 2, wherein the protein has a τm⁵U modification activity.

The nucleic acid may be DNA or RNA, e.g., mRNA and so forth.

The vector may be a viral vector or a plasmid. The viral vector can be selected from known vectors, for example, from the group consisting of retrovirus vectors, lentivirus vectors, adenovirus vectors, and adeno-associated virus vectors. The vector may be a self-replicating vector, for example, a plasmid, an extrachromosomal element, a minichromosome, or an artificial chromosome. Viral vectors are preferred among the preceding.

The MTO1-encoding nucleic acid is operably linked to at least one control sequence for the expression of this nucleic acid. The disposition of the MTO1-encoding nucleic acid is not particularly limited as long as it is under the control of a control sequence. A promoter sequence is a control sequence. The promoter may be a constitutive promoter, a tissue-specific promoter, or an inducible promoter. The constitutive promoter includes the SV40 promoter, cytomegalovirus (CMV) promoter, and so forth.

The control sequence may additionally comprise an operator or enhancer, a mRNA ribosome binding site, and other sites that regulate the initiation of transcription or translation.

### (Application of Water-Soluble Carbodiimides)

“Water-soluble carbodiimide” means a water-soluble compound that has a carbodiimide group (—N═C═N—) in the molecule. The water-soluble carbodiimide includes N-cyclohexyl-N′-β-(4-methylmorpholinium) ethylcarbodiimide (CMC), 1-ethyl-3-(3-dimethylaminopropyl) carbodiimide (EDC), 4-(4,6-dimethoxy-1,3,5-triazin-2-yl)-4-methylmorpholinium chloride (DMT-MM), and their salts.

A compound given by the following general formula can be formed when the water-soluble carbodiimide is contacted with mitochondrial tRNA^{Leu(UUR)}.

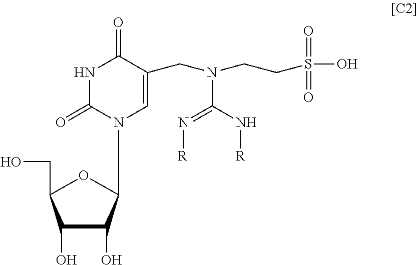

wherein R is a saturated or unsaturated hydrocarbon group, which may have a substituent, and the two R's may be the same as each other.

The “substituent” should be a functional group that can impart water solubility to the carbodiimide compound, but is not otherwise particularly limited; however, nitrogenous functional groups are preferred. Among these, nitrogenous functional groups that can form a quaternary salt are more preferred, for example, functional groups such as the N-alkylmorpholinyl group, trisubstituted amino group, and disubstituted amino group. The substituent on the amino group includes lower hydrocarbon groups, i.e., ethyl and methyl. R is preferably a substituent-bearing hydrocarbon group.

The “hydrocarbon group” in the “hydrocarbon group, which may have a substituent” includes C₁₋₁₉ hydrocarbon groups, e.g., alkyl groups, alkenyl groups, alkynyl groups, aryl groups, aralkyl groups, and so forth.

The alkyl group includes straight-chain or branched-chain C₁₋₆ alkyl groups, e.g., the methyl group, ethyl group, n-propyl group, isopropyl group, n-butyl group, isobutyl group, sec-butyl group, tert-butyl group, n-pentyl group, isopentyl group, sec-pentyl group, neopentyl group, n-hexyl group, and isohexyl group; and by cyclic C₃₋₁₄ alkyl groups, e.g., the cyclopentyl group and cyclohexyl group.

The alkenyl group includes straight-chain or branched-chain C₂₋₆ alkenyl groups, e.g., the allyl group, isopropenyl group, isobutenyl group, 2-pentenyl group, and 2-hexenyl group; and by cyclic C₃₋₁₄ alkenyl groups, e.g., the 2-cyclohexenyl group.

The alkynyl group includes straight-chain or branched-chain C₂₋₆ alkynyl groups, e.g., the propargyl group, 2-butynyl group, 3-butynyl group, 3-pentynyl group, and 3-hexynyl group.

The aryl group includes C₆₋₁₄ aryl groups, e.g., the phenyl group, 1-naphthyl group, 2-naphthyl group, biphenylyl group, and 2-anthryl group.

The aralkyl group includes phenyl-C₁₋₄ alkyl groups, e.g., the benzyl group, phenethyl group, and phenylpropyl group; and by C₇₋₁₉ aralkyl groups, e.g., the benzhydryl group and trityl group.

When the hydrocarbon group is an alkyl group, alkenyl group, or alkynyl group, the hydrocarbon group may be substituted by, for example, an alkylthio group (e.g., a C₁₋₄ alkylthio group, e.g., the methylthio group, ethylthio group, n-propylthio group, and isopropylthio group), a halogen atom (e.g., the fluorine atom, chlorine atom, bromine atom, and iodine atom), an alkoxy group (e.g., a C₁₋₆ alkoxy group, e.g., the methoxy group, ethoxy group, n-propoxy group, and isopropoxy group), an acyloxy group (e.g., a C₁₋₆ alkyl-carbonyloxy group, e.g., the acetyloxy group and n-propionyloxy group; a C₆₋₁₄ aryl-carbonyloxy group, e.g., the benzoyloxy group and naphthalenecarbonyloxy group), a nitro group, an alkoxycarbonyl group (e.g., a C₁₋₆ alkoxy-carbonyl group, e.g., the methoxycarbonyl group, ethoxycarbonyl group, n-propoxycarbonyl group, and isopropoxycarbonyl group), an alkylamino group (e.g., a mono- or di-C₁₋₆-alkylamino group, e.g., the methylamino group, ethylamino group, n-propylamino group, isopropylamino group, dimethylamino group, diethylamino group, methylethylamino group, and methylisobutylamino group), an alkoxyimino group (e.g., a C₁₋₆ alkoxyimino group, e.g., the methoxyimino group, ethoxyimino group, n-propoxyimino group, and isopropoxyimino group), or a hydroxyimino group. The number of substituents is not particularly limited, and may be, for example, 1 to 3. When there are two or more substituents, the individual substituents may be the same or may differ.

When the hydrocarbon group is an aryl group or aralkyl group, this hydrocarbon group may be substituted by, for example, an alkyl group (e.g., a straight-chain or branched-chain C₁₋₆ alkyl group, e.g., the methyl group, ethyl group, n-propyl group, isopropyl group, n-butyl group, isobutyl group, sec-butyl group, tert-butyl group, n-pentyl group, and n-hexyl group; and a cyclic C₃₋₆ alkyl group, e.g., the cyclohexyl group), an alkenyl group (e.g., a C₂₋₆ alkenyl group, e.g., the allyl group, isopropenyl group, isobutenyl group, 1-methylallyl group, 2-pentenyl group, and 2-hexenyl group), an alkynyl group (e.g., a C₂₋₆ alkynyl group, e.g., the propargyl group, 2-butynyl group, 3-butynyl group, 3-pentynyl group, and 3-hexynyl group), an alkoxy group (e.g., a C₁₋₆ alkoxy group, e.g., the methoxy group, ethoxy group, n-propoxy group, and isopropoxy group), an acyl group (e.g., a C₁₋₆ alkyl-carbonyl group, e.g., the formyl group, acetyl group, and propionyl group; a C₆₋₁₄ aryl-carbonyl group, e.g., the benzoyl group and naphthalenecarbonyl group), a nitro group, amino group, hydroxy group, cyano group, sulfamoyl group, mercapto group, a halogen atom (e.g., the fluorine atom, chlorine atom, bromine atom, iodine atom), or an alkylthio group (e.g., a C₁₋₄ alkylthio group, e.g., the methylthio group, ethylthio group, n-propylthio group, and isopropylthio group). The number of substituents is not particularly limited, and may be, for example, 1 to 5. When there are two or more substituents, the individual substituents may be the same or may differ.

A compound having the following structure can be formed when mitochondrial tRNA^{Leu(UUR)} is contacted with CMC as the water-soluble carbodiimide.

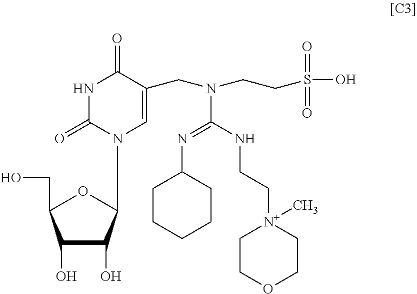

A compound having the following structure can be formed when mitochondrial tRNA^{Leu(UUR)} is contacted with CMC as the water-soluble carbodiimide.

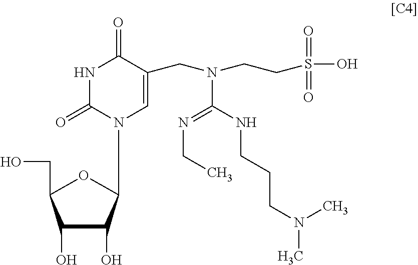

CMC, which is one of the water-soluble carbodiimides, has been used for the detection of pseudouridine (ψ) using a primer extension technique (Ofengand et al., 2001).

When a reverse transcription reaction is carried out on mitochondrial tRNA^{Leu(UUR)}, the reaction proceeds according to the course shown in FIG. 6

When τm⁵U modification is present, it is known that the reverse transcription reaction is as shown in FIG. 7, center, and that the reverse transcription reaction stops at a position corresponding to position 33 in mitochondrial tRNA^{Leu(UUR)} (Kirino et al., 2005). The present inventors discovered that when the reverse transcription reaction is carried out on a template of mitochondrial tRNA^{Leu(UUR)} in the presence of a water-soluble carbodiimide, e.g., CMC, the reverse transcription reaction can be inhibited by the formation of the compound given by the general formula indicated above.

Specifically, the reverse transcription reaction is stopped at a position corresponding to position 35 in the case of τm⁵U that has been derivatized by contact with water-soluble carbodiimide (FIG. 7, right). On the other hand, when unmodified U is present, the reverse transcription reaction proceeds unimpeded and does not stop at position 33, and the extension reaction is completed by the introduction of dideoxyguanosine triphosphate (ddGTP) at the C at position 32 (FIG. 7, left). While not intending to be bound by theory, it is believed that, due to the bulkiness of the water-soluble carbodiimide-derivatized τm⁵U, steric hindrance is produced to a degree that inhibits the reverse transcription reaction at the point of entry into the reverse transcriptase, and that, as a result, the reverse transcription reaction stops at a position corresponding to position 35, which is immediately before the τm⁵U modification.

The reverse transcription reaction can be carried out using a known method on a template of the tRNA^{Leu(UUR)} gene and using a reverse transcriptase, a primer, and dNTPs (nucleotide mixture). As used herein, “reverse transcription reaction” means a reaction mediated by a reverse transcriptase that carries out RNA-dependent DNA synthesis. The reverse transcription reaction is divided into an annealing reaction of the primer with the template followed by an extension reaction from the primer.

Primer extension reactions, RT-PCR, and so forth are examples of reactions that use a reverse transcription reaction.

The sample used in the reverse transcription reaction is obtained by preparing an RNA-comprising sample using a conventional method. The sample may originate not only from a healthy human or a subject that is suspected of having a disease(s) caused by reduction or deficiency in τm⁵U modification, but also from, for example, non-human vertebrates (e.g., monkeys, mice, rats, hamsters, guinea pigs, rabbits, cats, dogs, pigs, bovines, horses, sheep, birds, reptiles, amphibians, fish), protochordates, mollusks, and so forth. The sample includes blood, plasma, serum, bile, saliva, urine, tears, sweat, cerebrospinal fluid, and so forth.

In some instances the mitochondrial tRNA^{Leu(UUR)} derived from a subject suspected of having a disease(s) caused by reduction or deficiency in τm⁵U modification may have an A3243G mutation.

A primer that specifically hybridizes to mitochondrial tRNA^{Leu(UUR)} is used in order for the mitochondrial tRNA^{Leu(UUR)} present in the obtained RNA to function as the template. The primer may be an oligonucleotide complementary to the template. This oligonucleotide may be DNA, RNA, or a combination of DNA and RNA. The nucleotides constituting the oligonucleotide may be natural nucleotides or artificial nucleotides.

The length of the primer is not particularly limited as long as the ability to specifically hybridize to mitochondrial tRNA^{Leu(UUR)} can be retained, but is about 13 to 25 bases, for example, 13, 14, 15, 16, 17, 18, 19, 20, 21, 22, 23, 24, or 25 bases. When the primer is too short, a satisfactory hybridization efficiency is not obtained; when the primer is too long, the primer itself produces a secondary structure and/or may hybridize to another sequence, and the specificity is lost. The person skilled in the art can design the primer as appropriate.

A primer having the following sequence is an example of a DNA primer that is complementary to mitochondrial tRNA^{Leu(UUR)}.

### ACCTCTGACTGTAAAG (SEQ ID NO: 13)

The primer may be directly or indirectly labeled with a labeling substance, and the labeling substance includes radioactive substances such as ³²P, ³⁵S, ¹⁴C, and ³H; fluorescent substances such as FITC; enzymes such as alkali phosphatase; and particles such as a metal colloid. The position of labeling is not particularly limited.

The reverse transcriptase may be freely selected; for example, various wild-type reverse transcriptases can be used, including commercially available reverse transcriptases, e.g., SuperScript (registered trademark) reverse transcriptase, M-MuLV reverse transcriptase, AMV reverse transcriptase, and MultiScribe (registered trademark) reverse transcriptase.

The dNTP may be a mixture of 4 nucleotides (for example, dATP, dGTP, dCTP, dTTP) or may be 1 to 3 nucleotides complementary to the base sequence of the template. The dNTP may optionally comprise at least one dideoxynucleotide (ddATP, ddTTP, ddCTP, ddGTP) in order to stop primer extension at a desired position. When the reverse transcription reaction is carried out in the presence of dideoxyguanosine triphosphate (ddGTP), the extension reaction can be stopped by the insertion of ddGTP at the C at position 32 of mitochondrial tRNA^{Leu(UUR)}.

In the absence of ddGTP, the extension reaction does not stop at position 32 and in principle can proceed to the 5′-terminal of the tRNA itself, but it is thought that in actuality extension will stop at the water-soluble carbodiimide-derivatized pseudouridine (ψ) at position 27 or the 1-methylguanosine (m¹G) at position 9.

After the completion of the reverse transcription reaction, differences in the lengths of the primer extension products due to the presence/absence of inhibition by the water-soluble carbodiimide are determined. These differences can be detected by subjecting the primer extension products to electrophoresis.

Other steps may be carried out after the completion of the reverse transcription reaction, for example, a step in which the τm⁵U modification rate is measured. The τm⁵U modification rate can be measured by a known method.

An embodiment of the present invention based on inhibition of the reverse transcription reaction by the water-soluble carbodiimide is described as an example below.

In a third embodiment, there is provided a method for detecting τm⁵U modification of mitochondrial tRNA, wherein the method comprises a step of performing, in the presence of a water-soluble carbodiimide, a reverse transcription reaction on mitochondrial tRNA^{Leu(UUR)} contained in a sample derived from a subject.

In a preferred embodiment, the presence of τm⁵U modification is indicated when the reverse transcription reaction stops at a position corresponding to position 35, which is earlier than the τm⁵U at position 34 in mitochondrial tRNA^{Leu(UUR)}, or when the reaction stops at a position corresponding to position 33. On the other hand, the absence of τm⁵U modification is indicated when, in the presence of ddGTP, the reverse transcription reaction proceeds to the position corresponding to position 32. The absence of τm⁵U modification is indicated when, also in the absence of ddGTP, the reverse transcription reaction proceeds to or beyond position 32.

In a fourth embodiment, there is provided a method for screening for drugs that treat or prevent a disease(s) caused by reduction or deficiency in τm⁵U modification, wherein the method comprises a step of performing, in the presence of a water-soluble carbodiimide, a reverse transcription reaction on mitochondrial tRNA^{Leu(UUR)} contained in a sample derived from a subject that has been treated with a candidate substance, and a step of measuring the τm⁵U modification rate.

In a preferred embodiment, when the τm⁵U modification rate is increased in comparison to that for a sample where treatment with the candidate substance was not carried out, the candidate substance is selected as a drug that treats or prevents a disease(s) caused by reduction or deficiency in τm⁵U modification.

In a fifth embodiment, there is provided a method for the in vitro detection of a disease(s) caused by reduction or deficiency in τm⁵U modification of mitochondrial tRNA, wherein the method comprises a step of performing, in the presence of a water-soluble carbodiimide, a reverse transcription reaction on mitochondrial tRNA^{Leu(UUR)} contained in a sample derived from a subject.

In a preferred embodiment, it is indicated that the subject is not suffering from a disease(s) caused by reduction or deficiency in τm⁵U modification when the reverse transcription reaction stops earlier than a τm⁵U at position 34 of the mitochondrial tRNA^{Leu(UUR)} or stops at a position corresponding to position 33, or it is indicated that the subject is suffering from a disease(s) caused by reduction or deficiency in τm⁵U modification when the reverse transcription reaction progresses to or beyond the position corresponding to position 32 in the mitochondrial tRNA^{Leu(UUR)}.

In a sixth embodiment, there is provided a method for assessing whether a subject that has, or is suspected of having, a disease(s) caused by reduction or deficiency in τm⁵U modification is a target for treatment of the disease using, e.g., taurine and so forth, wherein this method comprises a step of performing, in the presence of a water-soluble carbodiimide, a reverse transcription reaction on mitochondrial tRNA^{Leu(UUR)} contained in a sample derived from a subject.

In a preferred embodiment, it is indicated that the subject can be a target for a treatment of the disease using, e.g., taurine and so forth, when the reverse transcription reaction stops earlier than a τm⁵U at position 34 of the mitochondrial tRNA^{Leu(UUR)} or stops at a position corresponding to position 33, or it is indicated that the subject can be a target for a treatment of the disease using, e.g., taurine and so forth, when the reverse transcription reaction progresses to or beyond the position corresponding to position 32 in the mitochondrial tRNA^{Leu(UUR)}.

### (Nucleic Acid Bonded to Water-Soluble Carbodiimide)

In a seventh embodiment, there is provided a compound having the following structure or a salt thereof:

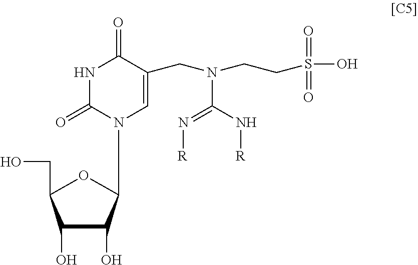

wherein R is a saturated or unsaturated hydrocarbon group, which may have a substituent, and the two R's may be the same as each other.

The “substituent” should be a functional group that can impart water solubility to the carbodiimide compound, but is not otherwise particularly limited; however, nitrogenous functional groups are preferred. Among these, nitrogenous functional groups that can form a quaternary salt are more preferred, for example, functional groups such as the N-alkylmorpholinyl group, trisubstituted amino group, and disubstituted amino group. The substituent on the amino group includes lower hydrocarbon groups, i.e., ethyl and methyl. R is preferably a substituent-bearing hydrocarbon group. R is defined as above.

The compound has the following structure when the water-soluble carbodiimide is CMC.

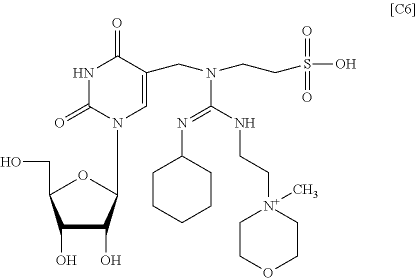

The compound has the following structure when the water-soluble carbodiimide is EDC.

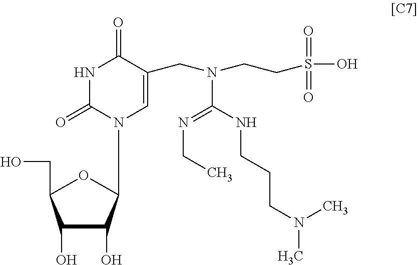

In an eighth embodiment, there is provided an inhibitor of a reverse transcription reaction on τm⁵U modification-comprising nucleic acid, wherein the inhibitor comprises a water-soluble carbodiimide as an active ingredient.

Besides τm⁵U modification-comprising nucleic acid, it is thought that the reverse transcription reaction inhibitor can also be used for other uridine derivatives that have a skeleton similar or analogous to τm⁵U modification-comprising nucleic acid.

The definition of this water-soluble carbodiimide is as above.

In a ninth embodiment, there is provided a kit comprising a reverse transcriptase and an inhibitor of a reverse transcription reaction on τm⁵U modification-comprising nucleic acid, wherein the inhibitor comprises a water-soluble carbodiimide as an active ingredient.

A freely selected reverse transcriptase can be used for the reverse transcriptase; for example, various wild-type reverse transcriptases can be used, including commercially available reverse transcriptases, e.g., SuperScript (registered trademark) reverse transcriptase, M-MuLV reverse transcriptase, AMV reverse transcriptase, and MultiScribe (registered trademark) reverse transcriptase, with SuperScript (registered trademark) III reverse transcriptase being preferred.

The present invention is more specifically described in the following examples, but the present invention is not limited to or by these.

### Examples

### 1. Construction of MTO1 Expression Vector

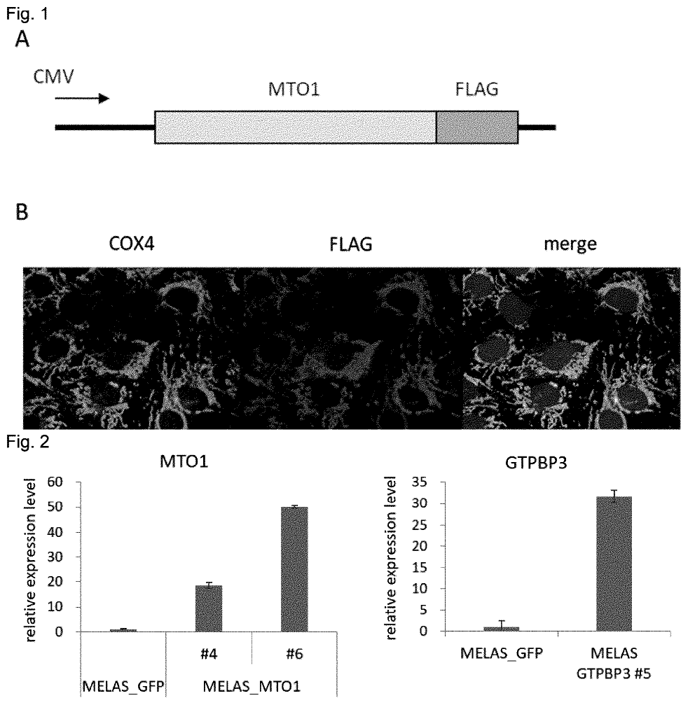

Homo sapiens-originating mitochondrial tRNA translation optimization 1 (MTO1) isoform a (NP_036255.2, NM_012123.4, MTO1 in the following) having the FLAG tag fused to the C-terminal (MTO1_FLAG in the following) was introduced into a lentivirus vector (FIG. 1A).

Using as template the vector pDEST_MTO1 (Asano et al., 2018) in which MTO1 cDNA was cloned, the MTO1 cDNA moiety was amplified by PCR (PCR reaction condition 1) using Fw primer 1 (SEQ ID NO: 3:

acctccatagaagacaccgactctagaggatccaccggtcgccaccATG
ttctacttccgaggctg)
and
Rv

primer 1 (SEQ ID NO: 4:

cggccgctttaTTTATCATCATCATCTTTATAATCTCCTCCtaactctc
tctcttgaagtctgtctg).

### PCR Reaction Condition 1

1×PCR buffer for KOD-Plus-Neo
0.2 mM dNTP
1.5 mM MgSO₄
0.25 pmol/μL Fw primer 1
0.25 pmol/μL Rv primer 1
8.4 ng/μL pDEST_MTO1
0.02 U/μL KOD-Plus-Neo

TABLE 1

| | | | | |
|---|---|---|---|---|
| 94° C. | 2 | min | | |
| 98° C. | 10 | sec | | |
| 58° C. | 30 | sec | {close oversize bracket} | ×24 cycle |
| 68° C. | 1.5 | min | | |
| 4° C. | | | | |

In addition, using pLenti CMV GFP Puro as template, the vector sequence required for cloning was amplified by PCR (PCR reaction condition 2) using Fw primer 2

(SEQ ID NO: 5: agtcagtccccgggggaggaGATTATAAAGATGA
TGATG)
and
Rv primer 2
(SEQ ID NO: 6: agtcagtcGATATCgaccggtggatcctctag).

### PCR Reaction Condition 2

1×PrimeSTAR buffer
0.2 mM dNTP
0.32 pmol/μL Fw primer 2
0.32 pmol/μL Rv primer 2
5 ng/μL pLenti CMV GFP Puro
0.05 U/μL PrimeSTAR DNA polymerase

TABLE 2

| | | | | |
|---|---|---|---|---|
| 94° C. | 2 | min | | |
| 98° C. | 10 | sec | | |
| 55° C. | 5 | sec | {close oversize bracket} | ×17 cycle |
| 72° C. | 10 | min | | |
| 4° C. | | | | |

After the PCR reaction, the template DNA was removed by adding DpnI (TAKARA) to each PCR product and incubating overnight at 37° C. This was followed by purification of the PCR product using a FastGene column (Nippon Genetics Co., Ltd.) and reaction (SLICE reaction conditions) using the SLICE method (Okegawa et al., 2015).

### SLICE Reaction Conditions (10 μL Reaction System)

50 mM Tris-HCl (pH 7.5)
10 mM MgCl₂
1 mM ATP
1 mM DTT
1 μL SLICE extract (S100 fraction of JM109)
10 ng linear vector
50 ng insert DNA

DH5a cells were then transformed with the reaction product and cultured overnight at 37° C. on LB agar culture medium comprising ampicillin. The grown colonies were inoculated onto liquid LB culture medium comprising ampicillin and the culture was shaken overnight at 37° C. Plasmid was extracted from the cultured cells, and a pLenti vector into which the cDNA of the target MTO1_FLAG (below, pLenti_MTO1_FLAG vector) had been inserted was confirmed by DNA sequence analysis. The MTO1_FLAG gene encoded in pLenti_MTO1_FLAG is transcribed under control by the CMV promoter (FIG. 1A).

### 2. Preparation of a Stable MTO1-Overexpressing Cell Line

Using a lentivirus system, the MTO1_FLAG gene was inserted into the genome of myoblasts derived from a MELAS patient (Sasarman et al., 2008) to construct a stable MTO1-FLAG-expressing cell line. To this end, a lentivirus expressing the MTO1_FLAG gene was first constructed using the following procedure. 2 μg of pLenti MTO1_FLAG, 7.5 μg of pMDLg/pRRE (Addgene #12251), 7.5 μg of pRSV/REV (Addgene #12253), and 5 μg of pMD2.G (Addgene #12259) were mixed with 176 μL of polyethyleneimine (PEI) and 2 mL of Opti-MEM (registered trademark) I Reduced Serum Medium (Gibco) and left for 20 minutes at room temperature. Transfection was carried out by adding the mixture to HEK293T cells (4×10⁶ cells) that had been seeded to a culture Petri dish (15 cm) on the previous day.

The HEK293T cells were cultured for 8 hours at 37° C. under 5% CO₂ in DMEM culture medium in the presence of 5% FBS, followed by exchange for fresh culture medium and collection of the culture medium after culture for 3 days. Fresh culture medium was added, culture was continued, and the culture medium was collected on the following day. The target packaged lentivirus is released into the culture supernatant. The recovered culture supernatant was filtered on a filter (0.22 μm) and the fraction passing through was subjected to ultracentrifugal separation for 1 hour at 35,000×g to recover the target lentivirus by sedimentation.

Myoblasts derived from a MELAS patient were transduced with the obtained lentivirus. A culture medium, which is provided by the addition of 1×penicillin-streptomycin solution (Wako), 10 mM taurine, and 50 μg/mL uridine to SkGM (registered trademark)-2 BulletKit (registered trademark) (Lonza), was used as the culture medium for culturing the myoblasts. After several days, the cells were subcultured in the medium comprising 2 μg/mL of puromycin for the selection of puromycin-resistant cells, and the grown cell colonies were isolated to obtain a stable expressing cell line. MTO1_FLAG expression within the cells and mitochondrial localization were confirmed by Western blotting and immunostaining for the FLAG tag (FIG. 1B). In addition, a stable GTPBP3-expressing cell line was also constructed for comparison. A stable green fluorescent protein (eGFP)-expressing cell line was constructed as a control cell line.

### 2.1 Confirmation of Expression by RT-PCR

The total RNA was extracted from each isolated cell line and the amount of mRNA was measured by RT-qPCR using the following protocol.

The admixed DNA was degraded by holding the reaction solution (10 μL), which contained 1 μg of total RNA, 1 μL of 10×Reaction Buffer [400 mM Tris-HCl (pH 8.0), 100 mM MgSO₄, 10 mM CaCl₂)] (Promega), and 1 μL of 1 U/mL RQ1 RNase-Free DNase (Promega), for 30 minutes at 37° C. After this, the reaction was stopped by adding 1 μL of Stop Buffer [20 mM EGTA (pH 8.0)] (Promega) and holding for 10 minutes at 65° C. cDNA was synthesized using a First Strand cDNA Synthesis Kit for RT-PCR (Roche), and 80 μL of ultrapure water was added. To 2.5 μL of the cDNA solution was added 7.5 μL of Premix [1 μL each of Fw primer and Rv primer, 48 μL of ultrapure water, and 100 μL of 2×KAPA SYBR FAST qPCR Master Mix (Roche)], and measurement was carried out under the following reaction conditions using a thermal cycler (Lightcycler 96, Roche).

TABLE 3
Reaction Conditions

| | | | | |
|---|---|---|---|---|
| 95° C. | 3 | min | | |
| 95° C. | 10 | s | | |
| 57° C. | 20 | s | {close oversize bracket} | ×40 |
| 72° C. | 1 | s | | |
| 95° C. | 5 | s | | |
| 65° C. | 60 | s | | |
| 97° C. | 1 | s | | |

### Primer Sequences

MTO1
(SEQ ID NO: 7)
Fw: AAACAAGCATATACCGGACAATC
Rv:
(SEQ ID NO: 8)
TTGATTCAATGGAGGGACAGTAT
GTPBP3
(SEQ ID NO: 9)
Fw: GTTTCACCGGTGAGGACTG
Rv:
(SEQ ID NO: 10)
TGTTTCCGCGTGGATAAGGT
GAPDH
Fw:
(SEQ ID NO: 11)
GAGCCAAAAGGGTCATCATC
Rv:
(SEQ ID NO: 12)
CCATCACGCCACAGTTTCC

Based on the RT-qPCR results for the MTO1- and GTPBP3-overexpressing cell lines, relative values were calculated versus the MTO1 and GTPBP3 mRNA in the MELAS_GFP cell line that is the control cell line. In addition, normalization with GAPDH mRNA as internal standard was also performed (FIG. 2). With regard to the MTO1 cell line, the steady-state level of MTO1 mRNA was increased 18.5- to 50.0-fold in comparison to the MELAS_GFP cell line. With regard to the GTPBP3 cell line, the mRNA was increased 31.7-fold in comparison to the MELAS_GFP cell line.

### 3. The CMC-PE Technique

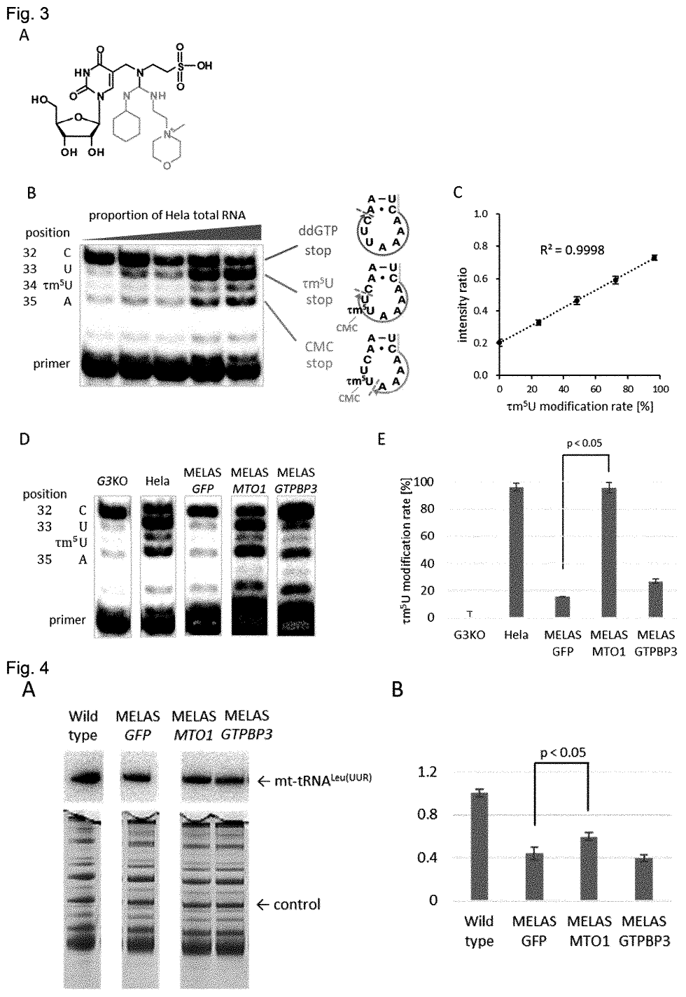

CMC (N-cyclohexyl-N′-β-(4-methylmorpholinium) ethylcarbodiimide) has been used for the detection of pseudouridine (ψ) using a PE technique (Ofengand et al., 2001). The present inventors discovered that, when τm⁵U has been derivatized with CMC (FIG. 3A), this stops the reverse transcription reaction at position 35 and position 33, and that with unmodified U the reverse transcription reaction proceeds and the extension reaction is completed by the introduction of dideoxyguanosine triphosphate (ddGTP) at the C of position 32 (FIG. 3B). In addition, quantitative measurement of a τm⁵U modification rate was made possible by quantitating the values of these three bands and using the values for the bands at position 33 and position 35 that participate in the total value of these bands. The band intensity ratio was calculated using the following formula.

band intensity ratio=(band intensity for position 33+band intensity for position 35)/(total value of band intensities for position 32, position 33, and position 35)

In the following, the PE technique using CMC is also referred to as the CMC-PE technique, but the same results as for CMC are also obtained when water-soluble carbodiimides other than CMC are used.

### CMC Derivatization of RNA

Using the acid guanidinium thiocyanate phenol chloroform extraction (AGPC) method (Chomczynski and Sacchi, 1987), the total RNA was extracted from the cells obtained as described above. A 15 μg fraction of the total RNA was dried up and dissolved in 15 μL of BEU buffer [50 mM bicine (pH 8.3), 4 mM EDTA (pH 8.0), 7 M urea]. 15 μL of BEU buffer in 0.34 M CMC-p-toluenesulfonate was added and mixed thoroughly, and a reaction was carried out for 20 minutes at 37° C. This was followed by the addition of 100 μL of stop solution [0.3 M NaOAc (pH 5.2), 0.1 mM EDTA (pH 8.0)] and 700 μL of ethanol and mixing, followed by recovery of the CMC-derivatized RNA by ethanol precipitation. Then, after rinsing with 70% ethanol, purification was carried out by dissolving the pellet in 100 μL of stop solution and reprecipitating with ethanol by adding 300 μL of ethanol. The purified RNA was vacuum dried and dissolved in 40 μL of 50 mM Na2CO₃ (pH 10.4) and a reaction was carried out for 4 hours at 37° C. to remove the CMC bonded to the uracil ring or guanine ring. To reaction solution was added 100 μL of stop solution for neutralization and the RNA was recovered by ethanol precipitation by adding 700 μL of ethanol and was dissolved in 10 μL of ultrapure water.

### Labeling of the 5′-Terminal of the Primer

³²P-labeling of the 5′-terminal of the primer was performed by carrying out a reaction for 1 hour at 37° C. in a reaction solution (10 μL) that contained 4 pmol (2 μL) of a DNA primer (SEQ ID NO: 13: ACCTCTGACTGTAAAG) complementary to mitochondrial tRNA^{Leu(UUR)}, 1 μL of [γ³²P]-ATP (PerkinElmer Inc.), 1 μL of 10× Protruding End Kinase Buffer (Toyobo), 0.5 μL of T4 Polynucleotide Kinase (Toyobo), and 5.5 μL of ultrapure water. This was followed by deactivation of the enzyme by heating for 15 minutes at 65° C., after which the ³²P-labeled DNA primer was submitted to gel filtration purification using a CENTRI-SEP spin column (Princeton Separations). The radioactivity was measured using Cerenkov light.

### Reverse Transcription Reaction

A solution (5 μL) comprising 0.75 μg of the CMC-derivatized total RNA, 0.05 pmol (approximately 50,000 cpm) of the ³²P-labeled DNA primer, 1 mM EDTA-NaOH (pH 8.0), and 10 mM Tris-HCl (pH 7.7) was heated for 2 minutes at 80° C., followed by gradual cooling to room temperature to hybridize the primer to the mitochondrial tRNA^{Leu(UUR)} in the total RNA. To this reaction solution were added 1.5 μL of 25 mM MgCl₂, 0.25 μL of d/dd NTP mix (1.5 mM dATP, 1.5 mM dTTP, 3.0 mM ddGTP), 2 μL of 5×FS buffer, 0.5 μL of SuperScript Ill reverse transcriptase (Invitrogen), and 0.75 μL of ultrapure water, an extension reaction was carried out for 1 hour at 55° C.; and incubated for 15 minutes at 70° C. to deactivate the enzyme. After the reaction, 0.5 μL of 4M NaOH was added and the RNA was degraded by heating for 5 minutes at 95° C. The reaction was neutralized by addition of 14.5 μL of Loading Solution (12.5 μL of 2×Loading Solution for Urea PAGE, 1 UL of 1 M Tris, and 1 μL of 1 M HCl) and incubation for 5 minutes at 65° C.

Quantitative Determination of the Amount of cDNA

The reaction product was separated using denaturing polyacrylamide gel electrophoresis (20% PAGE with 7 M urea, 20×20 cm², 0.35 mm thickness), the gel was exposed to an imaging plate (BAS-MS2040, Fujifilm) overnight (−30° C.), and the radiolabeled bands were detected using an image analyzer (FLA-7000, Fujifilm). The detected bands were quantitated using a MultiGauge.

### 4. Detection of τm⁵U Modification Using the CMC-PE Technique

In order to examine the quantitative character of the CMC-PE technique, CMC-PE was carried out using RNA extracted from Hela cells and from GTPBP3 knockout cells (G3KO) (FIG. 3B). It has been demonstrated by LC/MS analysis of mitochondrial tRNA^{Leu(UUR)} isolated and purified from each of these cells that the τm⁵U modification rates in G3KO cells and in Hela cells are 0% and 96.3%, respectively. Based on this, and using samples provided by mixing these two types of total RNAs at ratios of 0:1, 1:3, 2:2, 3:1, and 1:0, the band intensity ratio and the τm⁵U modification rate were associated by constructing a calibration curve between the band intensity ratio calculated by carrying out CMC-PE according to the method described above and the τm⁵U modification rate calculated from the mixing ratio of the two samples (FIG. 3C). Linearity in modification rate measurement using the PE technique was confirmed from the calibration curve.

The τm⁵U modification rate was quantitatively determined based on this calibration curve for the stable MTO1-expressing cell line, stable GTPBP3-expressing cell line, and stable eGFP-expressing cell line. It was found that, versus a τm⁵U modification rate of 15.5% in the stable eGFP-expressing cell line, the τm⁵U modification rate was increased to 26.7% in the stable GTPBP3-expressing cell line and to 95.3% in the stable MTO1-expressing cell line (FIGS. 3D, 3E).

5. Analysis of Steady-State Level of MELAS Mutant Mitochondrial tRNA Using Northern Blotting

Then, Northern blotting analysis was performed to investigate whether the steady-state level of MELAS mutant mitochondrial tRNA is changed by overexpression of MTO1 in MELAS cells. 5S rRNA was used for the control.

5 μg of total RNA was separated by denaturing polyacrylamide gel electrophoresis (10% PAGE with 7 M urea). A nylon membrane (Hybond-N+, GE Healthcare) was immersed in TBE buffer (44.5 mM Tris, 44.5 mM borate, 2 mM EDTA), the gel was stacked on this, filter paper immersed in TBE was stacked four above and four below this, a voltage of 1.5 mA/cm² was applied, and electroblotting was performed for 35 minutes. The membrane was then dried and was irradiated twice with UV at 120 mJ/cm² to crosslink the RNA to the membrane. The membrane was immersed in a hybridization buffer (PerfectHyb (registered trademark) Plus, Sigma) and was held for at least 30 minutes at 50° C. DNA probes complementary to, respectively, mitochondrial tRNA^{Leu(UUR)} and 5S rRNA (mitochondrial tRNA^{Leu(UUR)}: TGTTAAGAAGAGGAATTGAACCTCTGACTG (SEQ ID NO: 14), 5S rRNA: GGGTGGTATGGCCGTAGAC (SEQ ID NO: 15)) were labeled with ³²P using the same procedure as for the primer in the CMC-PE method and were added to the hybridization buffer and incubated overnight at 50° C.

The membrane was lightly washed with 1×SSC [150 mM NaCl, 15 mM sodium citrate (pH 7.0)] and then shaken for 10 minutes with 1×SSC 3 times. Printing to an imaging plate was carried out for about 1 hour and the radiolabeled bands were detected using an image analyzer.

As previously reported (Yasukawa et al., 2000), the steady-state level of mitochondrial tRNA^{Leu(UUR)} was significantly reduced in a stable eGFP-expressing cell line (MELAS myoblasts) in comparison to a wild-type cell line (FIGS. 4A and 4B). In comparison to the stable eGFP-expressing cell line, it was found that the steady-state level of mitochondrial tRNA^{Leu(UUR)} was not changed in the stable GTPBP3-expressing cell line, but was significantly increased in the stable MTO1-expressing cell line (FIGS. 4A and 4B). Based on these results, it was found that not only does the overexpression of MTO1 have the effect of increasing the τm⁵U modification rate of MELAS mutant mitochondrial tRNA^{Leu(UUR)}, it also has the effect of increasing the steady-state level.

### 6. Evaluation of the Protein Synthesis Capability by a Pulse-Labeling Technique

The mitochondrial protein synthesis was evaluated by stopping cytoplasmic translation reactions using the antibiotic emetine and carrying out pulse-labeling with [³⁵S]-methionine and -cysteine to specifically label mitochondrial translation products.

Myoblasts from healthy individuals, the stable eGFP-expressing cell line, and the stable MTO1-overexpressing cell line were seeded at 4.0×10⁵ cells to collagen-coated 6-well plates and were cultured overnight at 37° C. The culture medium was removed; washed with PBS twice; and replaced with DMEM-high glucose, no glutamine, no methionine, no cystine (#210313024, Gibco) comprising 10% dialyzed FBS, 10 mM taurine, and 2 mM L-glutamine; and cultured for 10 minutes at 37° C. Emetine (final concentration=50 μg/mL) was added to this; cultured at 37° C. for 10 minutes; this was followed by the addition of 0.2 mCi [³⁵S] methionine and cysteine (EXPRE³⁵S³⁵S Protein Labeling Mix, [³⁵S] methionine/cysteine, PerkinElmer, Inc.); and cultured for an additional 1 hour. The medium was changed to DMEM-high glucose (D5796, Sigma-Aldrich) comprising 10% dialyzed FBS and 10 mM taurine and cultured at 37° C. for 10 minutes. The culture medium was removed, washing with PBS was done twice, and the cells were collected with 50 μL of *Lysis buffer. The lysates were shaken for 30 minutes at 4° C. and 13,000 rpm, centrifuged for 20 minutes at 4° C. and 15,000 rpm, and the supernatant was collected.

*Lysis buffer: RIPA+EDTA buffer [50 mM Tris-HCl (pH 7.5), 150 mM NaCl, 1% NP-40, 0.5% sodium deoxycholate, 0.1% SDS, 1 mM EDTA-NaOH (pH 8.0)], 1 mM DTT, and 1×protease inhibitor (complete Protease Inhibitor Cocktail (#04693116001, Roche) dissolved in 2 mL of ultrapure water to prepare 25-fold concentrated stock solution)]

SDS loading solution was added to 30 μg of total protein, followed by denaturation with SDS by incubating for 30 minutes at 40° C., and separation was carried out with Tricine PAGE. The gel was washed three times with ultrapure water and stained with CBB stain one ((#04543-51, Nacalai Tesque, Inc.) for 1 hour. After destaining by washing with ultrapure water several times, the gel was shaken for 10 minutes in an aqueous solution comprising 20% methanol and 2% glycerol. The gel was thoroughly dried for 2 hours at 60° C. using a gel dryer (AE-3750 RapiDry, ATTO) set to a temperature gradient of 1° C./min; this was exposed to an imaging plate for about 3 days and detection of the radiolabeled bands using an image analyzer.

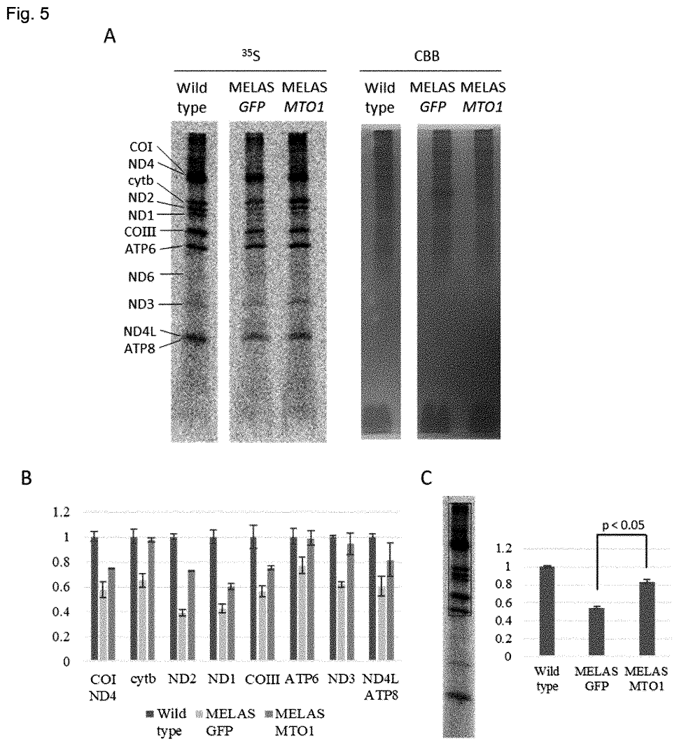

With the stable eGFP-expressing cell line, the mitochondrial protein synthesis capability was globally reduced in comparison to the wild type. It was shown that, for the stable MTO1-expressing cell line, the mitochondrial protein synthesis capability was increased relative to the stable eGFP-expressing cell line (FIG. 5).

The preceding results demonstrate that, by overexpression of MTO1 in MELAS patient cells, the τm⁵U modification rate of MELAS mutant mitochondrial tRNA^{Leu(UUR)} can be increased and mitochondrial function can be stimulated.

### LITERATURE

Suzuki, T., The expanding world of tRNA modifications and their disease relevance, Nature Reviews Molecular Cell Biology 22 (6), 375-392 (2021).
Suzuki, T., RNA modification and biological phenomenon, Journal of Japanese Biochemical Society 92 (3): 431-446 (2020).
Asano, K., Suzuki, T., Saito, A., Wei, F., Ikeuchi, Y., Numata, T., Tanaka, R., Yamane, Y., Yamamoto, T., Goto, T., Kishita, Y., Murayama, K., Ohtake, A., Okazaki, Y., Tomizawa, K., Sakaguchi, Y. and Suzuki, T. Metabolic and chemical regulation of tRNA modification associated with taurine deficiency and human disease. Nucleic Acids Res. 46, 1565-1583 (2018).
Okegawa, Y. and Motohashi, K. A simple and ultra-low cost homemade seamless ligation cloning extract (SLICE) as an alternative to a commercially available seamless DNA cloning kit. Biochem. Biophys. Rep. 4, 148-151 (2015).
Sasarman, F., Antonika, H., and Shoubridge, A., E. The A3243G tRNA^{Leu(UUR)} MELAS mutation causes amino acid misincorporation and a combined respiratory chain assembly defect partially suppressed by overexpression of EFTu and EFG2. Hum. Mol. Genet. 17, 3697-3707 (2008).
Kirino, Y., Goto, Y., Campos, Y., Arenas, J., & Suzuki, T. “Specific correlation between the wobble modification deficiency in mutant tRNAs and the clinical features of a human mitochondrial disease.” PNAS. 102, 7127-7132 (2005).
Ofengand, J., Del Campo, M. & Kaya, Y. Mapping pseudouridines in RNA molecules. Methods 25, 365-373 (2001).
Chomczynski, P. and Sacchi, N. Single-step method of RNA isolation by acid guanidinium thiocyanate-phenol-chloroform extraction. Anal Biochem 162, 156-159 (1987).
Yasukawa, T., Suzuki, T., Suzuki, T., Ueda, T., Ohta, S. and Watanabe, K. Modification Defect at Anticodon Wobble Nucleotide of Mitochondrial tRNAs^{Leu(UUR)} with Pathogenic Mutations of Mitochondrial Myopathy, Encephalopathy, Lactic Acidosis, and Stroke-like Episodes. Journal of Biological Chemistry 275, 4251-4257 (2000).

What is claimed is:

1. A pharmaceutical composition for the treatment or prevention of a disease(s) caused by reduction or deficiency in 5-taurinomethyluridine (τm⁵U) modification of mitochondrial tRNA, the composition comprising mitochondrial tRNA translation optimization 1 (MTO1) or nucleic acid encoding MTO1, wherein MTO1 is administered to a patient in an excess amount, or MTO1 is overexpressed in a patient to whom the nucleic acid has been administered.

2. The pharmaceutical composition according to claim 1, wherein a τm⁵U modification rate is increased by the administration of MTO1 or the nucleic acid, in comparison to that prior to the administration.

3. The pharmaceutical composition according to claim 2, wherein a τm⁵U modification rate is increased in comparison to the administration of excess GTP-binding protein 3 (GTPBP3) or in comparison to the overexpression of nucleic acid encoding GTPBP3.

4. The pharmaceutical composition according to any one of claims 1 to 3, wherein the nucleic acid encoding MTO1 has:
(i) nucleic acid consisting of a base sequence of SEQ ID NO: 1;
(ii) nucleic acid capable of hybridizing under stringent conditions with nucleic acid consisting of a base sequence complementary to the base sequence of SEQ ID NO: 1, and encoding protein having a τm⁵U modification activity;
(iii) nucleic acid consisting of a base sequence provided by deletion, substitution, or addition of one or a few bases in the base sequence of SEQ ID NO: 1, and encoding protein having a τm⁵U modification activity;
(iv) nucleic acid consisting of a base sequence having an at least 80% sequence identity with SEQ ID NO: 1, and encoding protein having a τm⁵U modification activity;
(v) nucleic acid consisting of a base sequence encoding protein consisting of the amino acid sequence of SEQ ID NO: 2;
(vi) nucleic acid capable of hybridizing under stringent conditions with nucleic acid consisting of a base sequence encoding protein consisting of the amino acid sequence of SEQ ID NO: 2, and encoding protein having a τm⁵U modification activity;
(vii) nucleic acid consisting of a base sequence encoding protein consisting of an amino acid sequence provided by deletion, substitution, or addition of one or a few amino acids in the amino acid sequence of SEQ ID NO: 2, with the protein having a τm⁵U modification activity; or
(viii) nucleic acid consisting of a base sequence encoding protein consisting of an amino acid sequence having a sequence identity of at least 80% with SEQ ID NO: 2, with the protein having a τm⁵U modification activity.

5. The pharmaceutical composition according to any one of claims 1 to 4, wherein the nucleic acid encoding MTO1 is contained in a vector.

6. The pharmaceutical composition according to claim 5, wherein the vector is a viral vector or a plasmid.

7. The pharmaceutical composition according to claim 6, wherein the viral vector is selected from the group consisting of retrovirus vectors, lentivirus vectors, adenovirus vectors, and adeno-associated virus vectors.

8. The pharmaceutical composition according to any one of claims 1 to 7, wherein the MTO1 or nucleic acid encoding MTO1 is administered systemically.

9. The pharmaceutical composition according to any one of claims 1 to 8, wherein the disease(s) caused by reduction or deficiency in τm⁵U modification is mitochondrial encephalomyopathy.

10. A vector comprising nucleic acid encoding MTO1.

11. A method of detecting τm⁵U modification of mitochondrial tRNA, the method comprising:
a step of performing, in the presence of a water-soluble carbodiimide, a reverse transcription reaction on mitochondrial tRNA^{Leu(UUR)} contained in a sample derived from a subject, wherein
the presence of τm⁵U modification is indicated when the reverse transcription reaction stops earlier than a τm⁵U at position 34 of the mitochondrial tRNA^{Leu(UUR)} or stops at a position corresponding to position 33, or
the absence of τm⁵U modification is indicated when the reverse transcription reaction progresses to or beyond a position corresponding to position 32.

12. The method according to claim 11, wherein, when τm⁵U modification is present, the reverse transcription reaction stops at a position of adenosine at position 35 or a position of uridine at position 33.

13. The method according to claim 11 or 12, wherein the reverse transcription reaction is a primer extension reaction using a reverse transcriptase.

14. The method according to any one of claims 11 to 13, wherein the method further comprises a step of measuring a τm⁵U modification rate.

15. The method according to any one of claims 11 to 14, wherein the mitochondrial tRNA^{Leu(UUR)} has an A3243G mutation.

16. The method according to any one of claims 11 to 15, wherein the water-soluble carbodiimide is selected from the group consisting of N-cyclohexyl-N′-β-(4-methylmorpholinium) ethylcarbodiimide (CMC), 1-ethyl-3-(3-dimethylaminopropyl) carbodiimide (EDC), 4-(4,6-dimethoxy-1,3,5-triazin-2-yl)-4-methylmorpholinium chloride (DMT-MM), and salts thereof.

17. A method of screening for drugs that treat or prevent a disease(s) caused by reduction or deficiency in τm⁵U modification, the method comprising:
a step of performing, in the presence of a water-soluble carbodiimide, a reverse transcription reaction on mitochondrial tRNA^{Leu(UUR)} contained in a sample derived from a subject that has been treated with a candidate substance; and
a step of measuring a τm⁵U modification rate,
wherein, when the τm⁵U modification rate is increased in comparison to that for a sample where treatment with the candidate substance has not been carried out, the candidate substance is selected as a drug that treats or prevents a disease(s) caused by reduction or deficiency in τm⁵U modification.

18. A method for in vitro detection of a disease(s) caused by reduction or deficiency in τm⁵U modification of mitochondrial tRNA, the method comprising:
a step of performing, in the presence of a water-soluble carbodiimide, a reverse transcription reaction on mitochondrial tRNA^{Leu(UUR)} contained in a sample from a subject, wherein
it is indicated that the subject is not suffering from a disease(s) caused by reduction or deficiency in τm⁵U modification when the reverse transcription reaction stops earlier than a τm⁵U at position 34 of the mitochondrial tRNA^{Leu(UUR)} or stops at a position corresponding to position 33, or
it is indicated that the subject is suffering from a disease(s) caused by reduction or deficiency in τm⁵U modification when the reverse transcription reaction progresses to or beyond a position corresponding to position 32 in the mitochondrial tRNA^{Leu(UUR)}.

19. A method for determining whether or not a subject that has or is suspected of having a disease(s) caused by reduction or deficiency in τm⁵U modification is to be a target for treatment with taurine, the method comprising:
a step of performing, in the presence of a water-soluble carbodiimide, a reverse transcription reaction on mitochondrial tRNA^{Leu(UUR)} contained in a sample derived from a subject, wherein
it is indicated that the subject can be a target for a treatment using taurine, when the reverse transcription reaction stops earlier than a τm⁵U at position 34 of the mitochondrial tRNA^{Leu(UUR)} or stops at the position corresponding to position 33, or
it is indicated that the subject can be a target for a treatment using taurine when the reverse transcription reaction progresses to or beyond the position corresponding to position 32 of the mitochondrial tRNA^{Leu(UUR)}.

20. A compound having a structure below or a salt thereof: 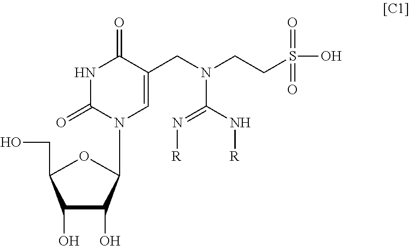
wherein R is a saturated or unsaturated hydrocarbon group, which may have a substituent, and two R's may be the same as each other.

21. An inhibitor of a reverse transcription reaction on τm⁵U modification-comprising nucleic acid, the inhibitor comprising a water-soluble carbodiimide as an active ingredient.

22. A kit comprising a reverse transcriptase and the reverse transcription reaction inhibitor according to claim 21.
